(19) United States
(12) Patent Application Publication
(10) Pub. No.: US 2022/0142798 A1
(43) Pub. Date: May 12, 2022

(54) INTRAVASCULAR FUNCTIONAL ELEMENT, SYSTEM HAVING A FUNCTIONAL ELEMENT, AND METHOD

(71) Applicant: Acandis GmbH, Pforzheim (DE)

(72) Inventors: Giorgio Cattaneo, Karlsruhe (DE); Andreas Schüssler, Pfinztal (DE); Martin Strobel, Pforzheim (DE); Christoph Janisch, Neuenbürg (DE)

(21) Appl. No.: 17/433,831

(22) PCT Filed: Feb. 19, 2020
(86) PCT No.: PCT/EP2020/054285

§ 371 Date: Aug. 25, 2021

(30) Foreign Application Priority Data
Feb. 26, 2019 (DE) 10 2019 104 827.3

(51) Int. Cl. A61F 2/90

(52) U.S. Cl. CPC A61F 2/90; A61F 2002/0081; A61F 2240/001; A61F 2210/0014

(57) ABSTRACT

The disclosure relates to an intravascular functional element, in particular an implant, more particularly a Stent, flow diverter, stent graft and intravascular occlusion device, having a radially self-expandable lattice structure which is tubular at least in some regions and which has a wire or a plurality of wires, wherein the wire/at least one of the wires includes a superelastic material, in particular a superelastic material of an alloy with the alloy elements nickel and titanium, wherein a mixed oxide layer is formed on the surface of the wire the wires with a layer thickness of 150 nm to 400 nm, in particular 200 nm to 350 nm, in particular 250 nm to 300 nm.

The invention relates to an intravascular functional element, a system having such a functional element and a method.

In medical technology, stents are generally produced by means of laser processes. However, braids made of Nitinol wires are also used for implants (e.g. stents or occluders). In contrast to stents produced by means of laser processes, in the case of wire braids, the wires slide over each other and therefore allow good deformation of the stent structures. In principle, (vascular) implants can be made of semi-finished products, such as metal sheets, precision pipes or wires.

For example, US 2004/0117001 A1 describes a method of producing a stent made of Nitinol. One aim of US 2004/0117001 A1 consists in reducing the nickel content in a layer near the surface in order to prevent nickel from being released from the layer, as the biocompatibility of the stent is impaired by this. A laser method is proposed for producing the stent. Following a cold processing stage, the stent is thermally treated and is then electropolished at temperatures under 20° C. For thermal oxidation the stent is exposed to hot steam at temperature of 150° C. for 12 hours. Through this, an oxidic surface with an Ni content of less than 2 percent by weight (wt %) and a layer depth of 10 nm should be achievable.

The known method has the drawback that in implants comprising wire braids, the oxide layer produced therewith, rapidly becomes worn.

EP 2 765 216 A1, which originates from the applicant, describes a production method in which the aforementioned problem is solved by a thermal treatment in a special salt bath. The production method, which has in the meantime been patented, is known by the proprietary name BlueOxide and is used for the surface treatment of braided stents or braided flow diverters, for example the flow diverter produced by the applicant under the proprietary name DERIVO.

Implants should be biocompatible, i.e. they should be as tolerable in the body as possible and exhibit good mechanical properties. For good biocompatibility it is beneficial, for example, if the smallest possible quantities of elements are released from the alloy composition of the implant, e.g. nickel from the lattice structure, and enter the blood flow. In addition, the compressibility of the implant should be as good as possible and in the implanted state in the vessel it should be as compliant as possible. This is the point at which the invention comes in, whereby its objective is to provide a functional element with good biocompatibility and mechanical properties. A further objective of the invention is to provide a system having such a functional element, as well as a method of producing a corresponding functional element.

In terms of the functional element, this task is solved by the subject matter of claim 1, in terms of the system, by the subject matter of claim 20 and in terms of the method, by the subject matter of claim 21.

More particularly the task is solved by an intravascular functional element having at least one radially self-expandable lattice structure which is tubular at least in sections and comprises one or a plurality of wires, wherein the wire/at least one of the wires, comprises a superelastic material, more particularly a superelastic material made of an alloy with the alloy elements nickel and titanium. The functional element can be an implant, more particularly a stent, flow diverter, stent graft and intravascular occlusion device. The invention is characterised in that the mixed oxide layer on the surface of the wire(s) is formed with a layer thickness of 150 nm to 400 nm, more particularly 200 nm to 350 nm, more particularly 250 nm to 300 nm.

The invention has various advantages. Surprisingly, it has been shown that a very thin mixed oxide layer particularly effectively reduces the diffusion of alloy elements into the body. It is assumed that the layer thickness in the claimed range results in a very dense and, in comparison with thicker layers, compact, i.e. less porous layer, and forms a particularly effective diffusion barrier. Furthermore, because of the good adhesion of the mixed oxide layer on the wire surface, the surface of the functional element according to the invention exhibits good mechanical properties. A further advantage consists in the fact that to form the mixed oxide layer, methods of surface treatment other than the usual electropolishing are used, e.g. mechanical polishing, through which the costs can be reduced.

Particular embodiments of the invention are set out the sub-claims.

Preferably, the quadratic roughness R_{q} of the wire(s) is from 0.02 μm to 0.5 μm, more particularly from 0.05 μm to 0.2 μm, more particularly from 0.06 μm to 0.1 μm.

The quadratic roughness R_{q} (rms roughness or root mean squared roughness) is calculated in a known manner from the mean of the deviation squares and corresponds to the “quadratic mean”. White light interferometry (WLI), which is known per se, and as a contactless optical measuring method utilises the interference of broadband light (white light) and thereby allows 3D profile measurements, can be used for roughness measurement, for example. 3D laser scanning microscopy can also be used, with which an adequate resolution is possible.

The roughness R_{q} of the wire(s) of 0.02 μm to 0.05 μm, which is increased in comparison with the known method according to EP 2 765 216 A1, allows the use of production methods other than the known electropolishing, which are optimised with regard to the intended homogenous properties of the wire(s) used to produce the functional element. For example, such production methods, more particularly mechanical polishing, can be used that result in an optimised wire diameter which has a positive effect on the crimping properties of the functional element.

Preferably, the quadratic roughness R_{q} of the wire(s) is from 0.02 μm to 0.5 μm, more particularly from 0.05 μm to 0.2 μm, more particularly from 0.06 μm to 0.1 μm. These ranges have proven to be particularly advantageous with respect to the wear properties and with respect to the homogenous properties of the wire(s) used for lattice structure.

In a preferred embodiment, the roughness, more particularly the quadratic roughness R_{q} in the circumferential direction of the wire(s) is greater than the roughness, more particularly the quadratic roughness R_{q} in the longitudinal direction of the wire(s). This anisotropic roughness profile improves the transporting properties of the functional element during implantation and can, for example, be adjusted in that the anisotropic roughness profile that sets in as a result of production of the wires by wire drawing is retained. This can be achieved, for example, in that the drawn wires are surface-treated in such a way that a greater roughness is set than through electropolishing, and the desired anisotropy is retained. The surface treatment involves deoxidation and/or mechanical polishing and/or chemical polishing. Mechanical polishing includes grinding, for example. Chemical polishing includes pickling, for example. Deoxidation is taken to mean the reduction of the oxide layer or changing the composition and/or properties of the oxide layer.

In general, the wire(s) is/are not electropolished.

Preferably, in the circumferential direction the roughness is greater by at least a factor of 1.5, more particularly by at least a factor of 2, more particularly by at least a factor of 5, more particularly by at least a factor of 10 than the roughness in the longitudinal direction. The quadratic roughness R_{q} in the circumferential direction of the wire(s) can be from 0.1 μm to 0.5 μm. The roughness, more particularly the quadratic roughness R_{q} in the longitudinal direction of the wire(s) can be from 0.02 μm to 0.1 μm, more particularly from 0.05 to 0.08 μm.

In a preferably preferred embodiment, the diameter of the wire(s) is essentially constant along the entire length of the wire. Particularly advantageously, the diameter of the wire(s) deviates by at most 10%, more particularly by at most 5%, more particularly by at most 3%, more particularly by at most 2%, more particularly by at most 1% from the average diameter of the respective wire(s). The essentially constant diameter of the wire(s) has the advantage that as a result of this, the mechanical properties of the functional element are formed evenly and homogenously along the lattice structure. In other words, the mechanical properties of the functional element along the longitudinal axis of the lattice structure and in the circumferential direction of the lattice structure are even and homogenous. In this way the crimping behaviour, for example, of the functional element is improved. Other properties in which the wire diameter plays a role, are also improved.

If the wire diameter deviates by at most 10%, more particularly by at most 5%, more particularly by at most 3%, more particularly by at most 2%, more particularly by at most 1% from the mean diameter of the wire/the respective wire, a particularly good improvement in the homogeneity of the properties of the lattice structure is achieved.

If the mean diameter of the wire(s) is from 30 μm to 60 μm, more particularly from 40 μm to 60 μm, a broad spectrum of applications for the intravascular functional element can be covered. The same applies for the nominal diameter of the lattice structure from 2.5 to 8 mm, more particularly from 3 mm to 5.5 mm, more particularly approximately 3.5 mm or, more particularly, approximately 4.5 mm.

The nominal diameter of the lattice structure essentially corresponds with the maximum diameter of application, i.e. the functional element manufacturer's recommended maximum diameter (intended use range). The difference between the maximum diameter recommended by the manufacturer of the functional element and the resting diameter, when no external forces are acting on the functional element consists in the wall thickness of the functional element. For example, the resting diameter of a functional element, more particularly a stent or a flow diverter, is approximately 4.7 mm with a nominal diameter of 4.5 mm and a wire thickness of 50 μm. The difference results from the quadruple wire thickness, as in a braided lattice structure, two wires arranged above each other have to be taken into account.

For improvement of the visibility under X-rays while largely retaining the mechanical properties, the wire/at least one of the wires can comprise a core material which is visible under X-rays, and a superelastic jacket material.

In a further particularly preferred embodiment, the wire is surface-treated, more particularly deoxidised, more particularly mechanically polished and/or chemically polished, more particularly pickled. If the lattice structure comprises a plurality of wires, the wires are each surface-treated, more particularly deoxidised, more particularly mechanically polished and/or chemically polished, more particularly pickled. If the wires are produced by drawing, for example, it is possible to adjust the desired roughness through the above surface treatment. The surface treatment allows greater roughness than through conventional electropolishing. It has also been shown that the wire diameter is essentially constant along its length if, during its production, the wire is surface-treated as explained above. In any event, particularly good homogenous properties of the functional element can be achieved through this.

The oxide layer formed on the wire surface is low in nickel and contains TiO₂, through which the corrosion behaviour and the biocompatibility of the functional element are improved. Producing the oxide layer as a mixed oxide layer in which at least titanium nitride and/or titanium oxynitride is/are present, increases the layer hardness as a result of which wear during stressing of the functional element, more particularly the implant, in the vessel is reduced. This advantage particularly comes into play in braids, such as braided stents, in which wires contact and slide on each other. In this way the quality of the functional element, more particularly of the implant, is improved, for example with regard to compliance in the vessel. Moreover, the coefficient of friction of the surface of the functional element, more particularly the implant, is reduced, which leads to improved sliding behaviour in the catheter. On the one hand the good sliding properties act between the wires themselves, through which the crimpability, i.e. the ability of the functional element to be compressed, is improved. On the other hand, the good sliding properties act between the wires and the inner wall of the catheter.

The thereby decreased pushing force, which is required for moving the functional element, more particularly the implant in the catheter, increases the safety, as the risk of blocking and damaging the functional element, more particularly the implant in the catheter, is reduced. The same applies for feed systems in which feeding of the functional element is not brought about through movement of the functional element itself, but through a relative movement between a part of the feed system and the functional element.

The embodiment in which in the longitudinal direction and in the circumferential direction, the lattice structure forms cells of intersecting wires, and in the circumferential direction has 16 cells to 32 cells, more particularly 20 cells to 28 cells, more particularly 24 cells, wherein the lattice structure has loops on a single axial end, is particularly suitable for flow diverters, in which it is important that the porosity of the functional element is set in such a way that the blood flow is guided in the vessel, for example for the treatment of aneurysms. An example of a flow diverter to which the aforementioned embodiment is applicable, is the applicant's flow diverter known under the proprietary name DERIVO. The loops each comprise a deflected wire.

The design features set out in sub-claims 14, 15 lead to an improvement in the homogenous properties or the visibility under X-rays of the functional element configured as a flow diverter.

The embodiment in which in the longitudinal direction and in the circumferential direction, the lattice structure forms cells from a single wire which is interwoven with itself, and in the circumferential direction has 6 cells to 16 cells, more particularly 8 cells to 12 cells, wherein lattice structure has loops on both axial ends, is suitable for a functional element configured as a braided stent, for example the stent produced by the applicant under the proprietary name ACCERO.

The design features set out in sub-claims 17 to 19 result in an improvement in the homo-genous properties or the visibility under X-rays of the functional element configured as a stent.

The number of cells on the circumference is determined by the number of wires or the number of wire segments which form the lattice structure. The number of wires or the number of wire segments is determined in that an imaginary interface intersects the lattice structure perpendicularly to its longitudinal axis. The interface thus extends in the radial direction with respect to the tubular lattice structure. The number of wire intersection points in the interface corresponds to the number of wires, in particular, individual wires or the number of wire segments of the lattice structure. In general, there are double as many wires or wire segments than cells present, wherein a first half of the wires or wire segments extends in first spiral direction and a second half of the wires or wire segments extends in a second spiral direction opposite to the first spiral direction so that the wires or wire segments intersect and form cells.

For example, 6 cells are formed on the circumference by 12 wires or wire segments, wherein 6 wires or wire segments circle around in a clockwise manner and 6 wires or wire segments in an anticlockwise manner.

In the case of a functional element with a braided lattice structure the cells are also known as meshes.

The lattice structure can be formed of a plurality of single wires, which extend without being deflected at both axial ends of the lattice structure, i.e. with free or open wire ends in each case, and to form the cells intersect each other, more particularly are interwoven with each other. It is also possible for the lattice structure to be formed of a single individual wire which is deflected at both axial ends of the lattice structure and forms loops there. The cells are formed by wire segments or by wire sections of the single wire, which extent on the circumference of the functional element in different spiral directions, and to form the cells intersect each other, more particularly are interwoven Furthermore, the lattice structure can be formed of a plurality of single wires, which are deflected at an axial end of the lattice structure and form intersecting, more particularly, braided wire segments. In this way the same number of cells on the circumference can be formed in the case of different functional elements by different numbers of single wires, depending on how many single wires are deflected at one or both axial ends of the lattice structure. If the lattice structure is formed by one, multiply deflected single wire, the number of cells on the circumference should be limited to a maximum of 16 cells, as otherwise the production of the lattice structure is very time-consuming.

The angle α, in particular the braid angle α, is, at least in sections, ≥45°, preferably ≥50°, preferably ≥55°, preferably ≥60°, preferably ≥65°, preferably ≥70°, preferably ≥75°, more particularly maximally 75°. With increasing braid angle, the flexibility of the braid increases.

In this context, the braid angle relates to the at-rest state of the of the functional element (in the expanded state). The braid angle is therefore determined in the relaxed state without the effect of external forces. This state can, for example, correspond to the manufacturing state. To determine the braid angle, the functional element is aligned straight in the axial direction. In this context, the braid angle is the designated as the angle formed between a wire and a perpendicular projection of the axis of rotation onto the circumferential plane of the lattice structure or the braid. The axis of rotation corresponds to the longitudinal axis of the braid or the lattice structure.

The wire cross-section is not restricted to a particular shape. Also possible are round, more particularly circular, elliptical, angular or other cross-section shapes of the wire. The functional element can, at least in sections, form a completely tubular braid, like a stent. Other applications such as occluders, flow diverters, filters or thrombectomy devices are possible.

The functional element, more particularly the implant, described above, can be part of a system comprising the functional element as well as a catheter, wherein a catheter inner diameter is preferably ≤0.8 mm, more preferably ≤0.7 mm, even more preferably ≤0.6 mm, even more preferably ≤0.5 mm, yet more preferably ≤0.4 mm. Independently disclosed and claimed in this connection, is also the use of a functional element (produced) as described above, more particularly as an implant for a catheter, and/or as a stent.

The wire braid, or in general, wire structure, can at least partially be produced from composite wires, wherein an inner core of the wire is made of a material that is visible under X-rays, such as platinum or tantalum.

It is known that AES is used for analysis of the elements of a material contained in a layer near the surface. Through successive removal of the layer by sputtering, the AES analysis of the respectively exposed layer surface produces a depth profile of the element distribution in the layer which is used for characterising the nitrogen content in relation to the oxygen content as well for detecting the course of concentration of the other elements such as Ni and Ti. The measured intensity of the respective element is determined in a known matter from the auger electrons emitted in the AES analysis through electronic bombardment.

To produce the intravascular functional element according to the invention that can be inserted into a hollow organ, a method is used in which the following steps are carried out:

Before producing a lattice structure of the functional element, a wire or plurality of wires, is/are surface-treated or a surface-treated wire or a plurality of surface-treated wires is/are provided. The surface treatment is carried out in such a way that the roughness values occurring during the mechanical production of the wires, more particularly through drawing, are less strongly reduced than in the hitherto usual electropolishing. This is achieved in that the surface treatment comprises deoxidation and/or polishing, more particularly grinding, and/or chemical polishing, more particularly pickling of the wire surface after mechanical production, more particularly after drawing. The lattice structure is then formed, more particularly braided, from the surface-treated wire(s). After the lattice structure has been formed, more particularly braided, an oxide layer is applied to the surface of the wire(s) with a layer thickness of 150 nm to 400 nm through thermal treatment.

For the provision of a wire metal body with a metallic surface, a preliminary treatment is carried out in which the oxide layer that is usually present on the wire surface is removed. This oxide layer with a thickness of 0.2 μm to 5 μm occurs during production of the wire when a thermal treatment is carried out to adjust the material properties of the wire. For removal of the oxide layer, the wire is surface-treated as described above. If the lattice structure comprises a plurality of wires, these are surface-treated before the lattice structure is produced. In the context of the method according to the invention, it is possible for the wires or the single wire to be surface-treated as a partial step. It is also possible that a wire, which is surface-treated in a separate process, or surface-treated wires is/are provided and processed into the functional element in accordance with the method.

With regard to the advantages of the functional element produced with the method according to the invention, reference is made to the above explanations.

On the metallic surface of the wire metal body, a first oxide layer can be formed, onto which the second mixed oxide layer is thermally applied. In the simplest case, the first oxide layer can be formed as a natural oxide layer, which comes about if the metallic surface of the wire metal body is exposed to the ambient air.

After transformation of the wire into a wire structure with at least one intersection, i.e. into a lattice structure or a lattice braid, thermal oxidation takes place in a salt bath. Through this, the surface is modified, more particularly passivated and hardened. As, at least in the boundary area close to the surface, the naturally formed oxide layer is low in nickel or is even nickel-free and therefore acts as a barrier with regard to the metal interface of the wire, the thermally formed oxide layer also only has a low Ni content or, at least in the boundary area close to the surface, is low in nickel or even nickel-free. Through the subsequent treatment in the salt bath, more particularly a salt bath containing nitrogen, a dense mixed oxide layer is produced on the naturally formed oxide layer which contains TiO₂. In addition, the mixed oxide layer contains portions of nitrogen, which is bound as titanium oxynitride and/or titanium nitride. Titanium oxynitride and/or titanium nitride result(s) from the salt bath, for example, when using an alkaline metal-nitrogen salt, more particularly potassium nitrate or sodium nitrite or a mixture of potassium nitrate and sodium nitrite. The thermally formed nitride, more particularly titanium oxynitride and/or titanium nitride, act(s) as a hardener which increases the layer hardness and improves the wear and friction behaviour of the functional element, more particularly implant.

The invention is not restricted to a special NiTi alloy, but can be used generally with the NiTi alloys that are customary in medical technology and from which intravascular functional elements, more particularly implants, are made, the surfaces of which are to be protected by an oxide layer.

Eligible as an alloy are, for example, various binary Ni-based compounds, such as NiTi alloys, more particularly nitinol (Ni 55 wt %, Ti 45 wt %) or various ternary compounds such as NiTiFe or NiTiNb of NiTiCr or quaternary alloys such as NiTiCoCr.

The wire can comprise at least 5 wt %, preferably at least 10 wt %, preferably at least 20 wt %, preferably at least 40 wt % nickel. The wire can also comprise at most 80 wt %, preferably at most 60 wt %, preferably at most 55 wt %, preferably at most 50 wt % nickel. The titanium content can preferably be at least 10 wt %, preferably at least 30 wt %, preferably at least 40 wt %, preferably at least 50 wt %. An upper limit for the titanium content can be 90 wt %, preferably at most 80 wt %, preferably 65 wt %, preferably 60 wt %, preferably at least 55 wt %.

The functional element, more particularly implant, is, for example, a braided stent or another implant, for example a flow diverter or a stent graft or intravascular occlusion device or an intravascular coil. The mixed oxide layer can comprise at least 10 wt %, more preferably at least 20 wt %, more preferably at least 30 wt %, more preferably at least 40 wt %, more preferably at least 50 wt % titanium oxynitride and/or titanium nitride.

The invention is explained as an example by way of an intravascular functional element in the form of a flow diverter, the lattice structure or lattice braid of which is made of a plurality of wires. The flow diverter has, at least in sections, a fully tubular lattice structure. In the radial direction, i.e. perpendicularly to the longitudinal axis of the lattice structure, the lattice structure is self-expandable. The lattice structure, or the wire forming the lattice structure, is made of a shape-memory material, more specifically an alloy with the alloy elements nickel and titanium, more particularly the alloy known under the proprietary name NITINOL. The surface of the wire has a quadratic roughness R_{q} which is at least 0.01 μm. The quadratic roughness R_{q} can, for example, be at least 0.0127 μm (0.5 μin), more particularly at least 0.0254 μm (1 μin). The maximum quadratic roughness R_{q} can be 0.08 μm, more particularly 0.0762 μm (3 μin), more particularly 0.0508 μm (1 μin).

The aforesaid quadratic roughness Rq can be adjusted by the aforementioned surface treatment of the wire(s).

In addition to the aforementioned roughness, the functional element is characterised by a mixed oxide layer on the surface of the wire(s), wherein the layer thickness of the mixed oxide layer is at least 150 nm, more particularly at least 200 nm. In the example, the oxygen content increases up to a layer thickness of approximately 25 nm starting from the surface (0 nm) and remains constant up to approximately 50 nm. Up to a layer depth of approximately 150 mm the oxygen content continuously decreases. Inversely, the nickel content increases with increasing layer thickness and reaches a maximum in the range of approximately 150 nm. This is similar for the titanium portion. In the layer thickness range of approximately 100 nm the oxygen and nickel curves overlap. In this range, mixed oxides are formed, such as TiO₂ and at least one nitride, more particularly titanium oxynitride and/or titanium nitride.

In a further example, the thickness of the mixed oxide layer is approximately 450 nm. The maximum thickness of the mixed oxide layer is 450 nm, more particularly maximally 300 nm.

The aforesaid quadratic roughness R_{q} allows or facilitates the formation of a mixed oxide layer of the aforementioned thickness. Adjusting the aforesaid roughness R_{q} by mechanical polishing has the further advantage that the respective wire essentially has a constant diameter. The wire diameter deviates from the mean wire diameter by a maximum of 10%, more particularly by a maximum of 5%, more particularly by a maximum of 3%. The essentially constant wire diameter means that the wire properties, more particularly the mechanical wire properties are homogenous along the entire length or the entire circumference of the lattice structure. The homogenous mechanical properties facilitate the crimpability and improve the behaviour of the functional element in the vessel.

The wire is made of a binary NiTi allow, such as Nitinol. Other alloys containing NiTi are possible. The modification of the surface is embodied by the treatment in the salt bath, which is responsible for adjusting the nitrogen concentration in the TiO₂ mixed oxide layer.

In the first step, the basic element of the functional element, namely the wire, is surface-treated, wherein the oxide layer formed after the thermal treatment for conditioning the wire is removed. A homogenous natural oxide layer forms spontaneously on the surface-treated wire through contact with the ambient air.

In the second step, the lattice structure, for example a stent, or in the case of a plurality of several surface-treated wires, a flow diverter, is braided from the surface-treated wire. In the third step the functional element is thermally treated in the salt bath to increase the layer thickness. The above principle for manufacturing the functional element is disclosed and claimed both in connection with, and also generally, i.e. independently of, the specific embodiments.

1-23. (canceled)

24. An intravascular functional element comprising:
a radially self-expandable lattice structure that is tubular at least in sections, the lattice structure including a wire having a superelastic material of an alloy with alloy elements nickel and titanium, and
wherein a mixed oxide on a surface of the wire is formed with a layer thickness of 150 nm to 400 nm.

25. The functional element according to claim 24, wherein a quadratic roughness R_{q} of the wire is from 0.02 μm to 0.5 μm.

26. The functional element according to claim 24, wherein a quadratic roughness R_{q} in a circumferential direction of the wire is greater than the quadratic roughness R_{q} in a longitudinal direction of the wire.

27. The functional element according to claim 26, wherein, in the circumferential direction, the roughness is greater by at least a factor of 1.5 than the roughness in the longitudinal direction.

28. The functional element according to claim 26, wherein the quadratic roughness R_{q} in the circumferential direction of the wire is from 0.1 μm to 0.5 μm.

29. The functional element according to claim 26, wherein the quadratic roughness R_{q} in the longitudinal direction of the wire is from 0.02 μm to 0.1 μm.

30. The functional element according to claim 24, wherein a diameter of the wire is substantially constant along an entire wire length and deviates by at most 10% from a mean diameter of the wire.

31. The functional element according to claim 30, wherein the mean diameter of the wire is 30 μm to 60 μm.

32. The functional element according to claim 24, wherein a nominal diameter of the lattice structure is 3 mm to 5.5 mm.

33. The functional element according to claim 24, wherein the wire comprises a core material visible under X-rays and a superelastic jacket material.

34. The functional element according to claim 24, wherein the wire is surface-treated.

35. The functional element according to claim 24, wherein the mixed oxide layer comprises TiO₂ and at least one nitride.

36. The functional element according to claim 24, wherein the lattice structure in a longitudinal direction and in a circumferential direction forms cells of intersecting wires and in the circumferential direction has 16 cells to 32 cells, wherein the lattice structure has loops on a single axial end.

37. The functional element according to claim 36, wherein a mean diameter of the wire is from 35 μm to 50 μm.

38. The functional element according to claim 36, wherein a core material of the wire is one of a platinum or a platinum alloy, and wherein a platinum portion is from 10% to 40%.

39. The functional element according to claim 24, wherein the lattice structure in a longitudinal direction and in a circumferential direction forms cells of a single wire interwoven with itself, wherein the cells of the wire in the circumferential direction are between 6 cells to 16 cells, and wherein the lattice structure has loops on both axial ends.

40. The functional element according to claim 39, wherein for a nominal diameter of the lattice structure of 2.5 to 3.5 mm, a mean diameter of the wire is from 40 μm to 55 μm, and wherein for the nominal diameter of the lattice structure of 3.5 to 8 mm, the mean diameter of the wire is from 45 μm to 65 μm.

41. The functional element according to claim 39, wherein a core material of the wire is one of a platinum or a platinum alloy, and wherein a platinum portion is from 20% to 40%.

42. The functional element according to claim 39, wherein a braid angle α of the lattice structure between the wire and a longitudinal axis extending in the longitudinal direction of the lattice structure is at least in sections 60° and 70°.

43. A system comprising:
an intravascular functional element having a radially self-expandable lattice structure;
a tubular element in which the functional element is arranged; and
a transport wire on which the functional element is fastened, wherein a quadratic roughness R_{q} in a circumferential direction of the wire is greater than the quadratic roughness R_{q} in a longitudinal direction of the wire, and wherein an inner diameter of the tubular element is at most 0.8 mm.

44. A method of producing an intravascular functional element adapted for insertion in a hollow organ, comprising:
providing a surface-treated wire;
forming a radially self-expandable lattice structure from the surface-treated wire, the lattice structure having a tubular form at least in sections, the wire having a superelastic material of an alloy with alloy elements nickel and titanium; and
applying an oxide layer to a surface of the wire with a layer thickness of 150 nm to 400 nm by way of a thermal treatment.

45. The method according to claim 44, wherein a temperature of the thermal treatment is between 450° and 600°.

46. The method according to claim 44, wherein after the thermal treatment, the lattice structure is quenched.
